(19) United States
(12) Patent Application Publication
(10) Pub. No.: US 2004/0193118 A1
(43) Pub. Date: Sep. 30, 2004

(54) Valved hub for a catheter

(76) Inventor: Brian J. Bergeron, Worcester, MA (US)

Correspondence Address: Fay Kaplun & Marcin, LLP, 150 Broadway, Suite 702, New York, NY, 10038, US

(21) Appl. No.: 10/396,727

(22) Filed: Mar. 25, 2003

(51) Int. Cl. A61M 5/00

(52) U.S. Cl. 604/247; 604/500

(57) ABSTRACT

A valved catheter assembly is designed to be used to minimize patient discomfort and to prevent infection at an entry site. The valved catheter assembly includes a hub including a slit valve disposed therein and distal tubing extending in a distal direction from the hub when the valved catheter assembly is inserted into a body of a patient. The valved catheter assembly is free of proximal tubing extending in a proximal direction from the hub.

### TECHNICAL FIELD

This invention relates to a valved catheter. More particularly, this invention relates to a catheter including a valve disposed within a hub.

### BACKGROUND INFORMATION

Intravenous catheters are long, soft tubular members that deliver fluids to a patient's blood stream. A peripherally inserted central catheter (PICC) is a particular type of intravenous catheter that is inserted into a patient's vein at an entry site, such as, for example, an entry site located near the bend of the patient's right arm. A medical professional, such as a physician or nurse, guides a distal tip of the PICC through the patient's vein and positions the distal tip at a predetermined cardiac site, such as, for example, the right atrium of the patient's heart.

Once properly positioned within the patient's body, a portion of the PICC extends from the entry site and is external to the patient's body. This portion of the PICC is generally about five centimeters to about twenty centimeters long and is accessible to the medical professionals to provide the patient with intravenous treatments, such as, for example, to deliver medications, fluids, nutritional supplements, blood and blood products, as well as to withdraw blood for laboratory tests.

A hub disposed on the tubular member of the PICC near the entry site secures the external portion of the PICC to the patient's body. Typically, a clamp positioned on the external portion of the PICC is used to prevent leakage in between intravenous treatments.

### SUMMARY OF INVENTION

The present invention generally relates to peripherally inserted central catheters. Devices according to the invention are typically used to deliver fluids directly to a patient's blood stream. It is an object of this invention to provide a device that allows fluid access to a predetermined cardiac site while, at the same time, minimizes patient discomfort and infection at an entry site. It is another aspect of the invention to provide a device that extends externally from the entry site less than about 3.81 centimenters.

In one aspect, the invention relates to a valved catheter assembly. The valved catheter assembly includes a catheter and a hub. The catheter includes a distal end including an opening, a proximal end, and at least one lumen extending therethrough. The hub is attached to the proximal end of the catheter. The hub includes a distal portion defining a lumen extending therethrough, a proximal portion also defining a lumen extending therethrough and including a luer proximal end. The hub also includes a flexible, thin disk including a slit. The flexible, thin disk is disposed within the hub such that a portion of the flexible, thin disk is in contact with the proximal portion of the hub and the slit is substantially perpendicular to a longitudinal axis of the hub. The valved catheter assembly is sized such the catheter spans a distance commensurate with a distance from a predetermined cardiac site to an entry site in the patient's skin.

Embodiments of this aspect of the invention can include the following features. In one embodiment, the distal portion of the hub includes a suture wing. In some embodiments, the valved catheter assembly further includes a transparent dressing positioned over the hub and entry site. In other embodiments, the hub is secured to the patient's skin. In one embodiment, the hub is situated immediately adjacent to the entry site.

In another aspect, the invention relates to a valved catheter assembly including a hub and distal tubing. The hub includes a distal end, a luer proximal end, and a valved disposed therebetween. The distal tubing extends in a distal direction from the distal end of the hub and the leur proximal end of the hub is free of proximal tubing.

Embodiments of this aspect of the invention can include the following features. In one embodiment, the distal end of the hub includes a suture wing. In some embodiments, the hub is secured to a patient's skin. In other embodiments, the valved catheter assembly further includes a transparent dressing positioned over the hub and entry site.

In another aspect, the invention relates to a valved catheter assembly including a hub and a catheter. The hub includes a slit valve disposed therein. The catheter is disposed distal to the hub and in fluid communication with the hub. The catheter includes a distal end, a proximal end and a tubular element extending between the distal and proximal ends and wherein the tubular element is free of a clamp disposed thereon.

Embodiments of this aspect of the invention can include the following features. In one embodiment, the distal end of the hub includes a suture wing. In some embodiments, the hub is secured to a patient's skin. In other embodiments, the valved catheter assembly further includes a transparent dressing positioned over the hub and entry site.

In general, in another aspect, the invention relates to a method of delivering fluids to a patient. The method, according to this aspect of the invention, includes providing a valved catheter assembly described above, inserting the valved catheter assembly into the patient's body through an entry site, and attaching the luer proximal end to tubing adapted for delivering fluids to the valved catheter assembly. In one embodiment of this aspect of the invention, the method can further include a step of measuring a distance spanning between the patient's right atrium and the entry site and then trimming the distal tubing to a length substantially equal to said distance prior to inserting the valved catheter assembly into the patient's body.

The foregoing and other objects, aspects, features, and advantages of the invention will become more apparent from the following description and the claims.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a schematic view of one embodiment of a prior art peripherally inserted central catheter.

FIG. 2 is a schematic view of the prior art peripherally inserted central catheter of FIG. 1 inserted within a patient's body.

FIG. 3 is a schematic view of one exemplary embodiment of a peripherally inserted central catheter according to the invention.

FIG. 4 is an exploded view of a portion of the peripherally inserted central catheter labeled A in FIG. 3.

FIG. 5 is a schematic view of one exemplary embodiment of an internal valve located within the portion of the peripherally inserted central catheter shown in FIG. 4.

FIG. 6 is a schematic view of the peripherally inserted central catheter of FIG. 3 inserted within a patient's body.

### DESCRIPTION

Intravenous catheters are used to transport fluids, such as, medications, nutritional supplements, and blood to a patient's blood stream. Peripherally inserted central catheters (PICC) are a type of intravenous catheters that are inserted into a vein within the patient's right arm. Generally, the PICC is used for multiple intravenous treatments and once positioned within the patient body, a PICC can be left in place up to about six months. In between treatments a clamp positioned on an external portion of the PICC prevents leakage from the PICC.

FIG. 1 shows an illustrative prior art PICC 1. The prior art PICC 1 includes a catheter (tubular member) 2, a hub 8, and proximal tubing 18. The catheter 2 is the portion of the PICC 1 that is insertable into a patient's body. The hub 8 and the proximal tubing 18 remain external to a patient's body while the prior art PICC 1 is in use.

The catheter 2 extends between a distal end 4 to a proximal end 6. The distal end 4 is open and is in fluid communication with a lumen extending within the catheter 2. The proximal end 6 is also in fluid communication with the lumen and is secured to a hub 8. The hub 8 includes a distal portion 10 and a proximal portion 12. Disposed between the distal portion 10 and proximal portion 12 are a pair of suture wings 14 that help to secure the hub 8 to the patient's body via suture openings 16.

Extending from the proximal portion 12 of the hub 8 is proximal tubing 18. The proximal tubing 18 has a length long enough to support a clamp 20 and generally has a length ranging between about 3.81 centimeters (1.5 inches) to about 15.24 centimeters (6 inches). The clamp 20 when activated collapses a lumen extending through the proximal tubing 18, thereby limiting leakage from the prior art PICC 1.

At a proximal end 22 of the proximal tubing 18 is a luer end cap 24. During medical treatments the lumen extending through the proximal tubing 18 is open and the luer end cap 24 is connected to a fluid introduction source, such as, for example, an intravenous bag, a cannula, or a syringe.

Referring to FIG. 2, the prior art PICC 1 is introduced into a patient's body 30 through an entry site 34 located just above a bend in the patient's right arm. Other entry sites, such as, for example, the groin, neck, and back of the patient are also available sites to introduce the prior art PICC 1. A medical professional guides the distal tip 4 of the catheter 2 from the entry site 34 through the patient's vein until the distal tip 4 is positioned within the right atrium of the patient's heart 32. The medical professional then secures the prior art PICC 1 to the patient's body via the hub 8. Once secured, the external portion of the PICC 1 (the hub 8, proximal tubing 19, clamp 20, and luer end cap 24) extends about 5.08 centimeters (2 inches) to about 20.32 centimeters (8 inches) from the entry site 34.

One of the problems with the prior art PICC 1 is that the proximal tubing 18 in combination with the clamp 20 and luer end cap 24 can be irritating to the patient's skin. Also, the length of the external portion (about 2 inches to about 8 inches) is awkward and inhibits the patient's free movement. Another problem with the prior art PICC 1 is that the clamp 20 does not satisfactorily close the lumen extending through the proximal tubing 18, resulting in leakage and infection at the entry site 34.

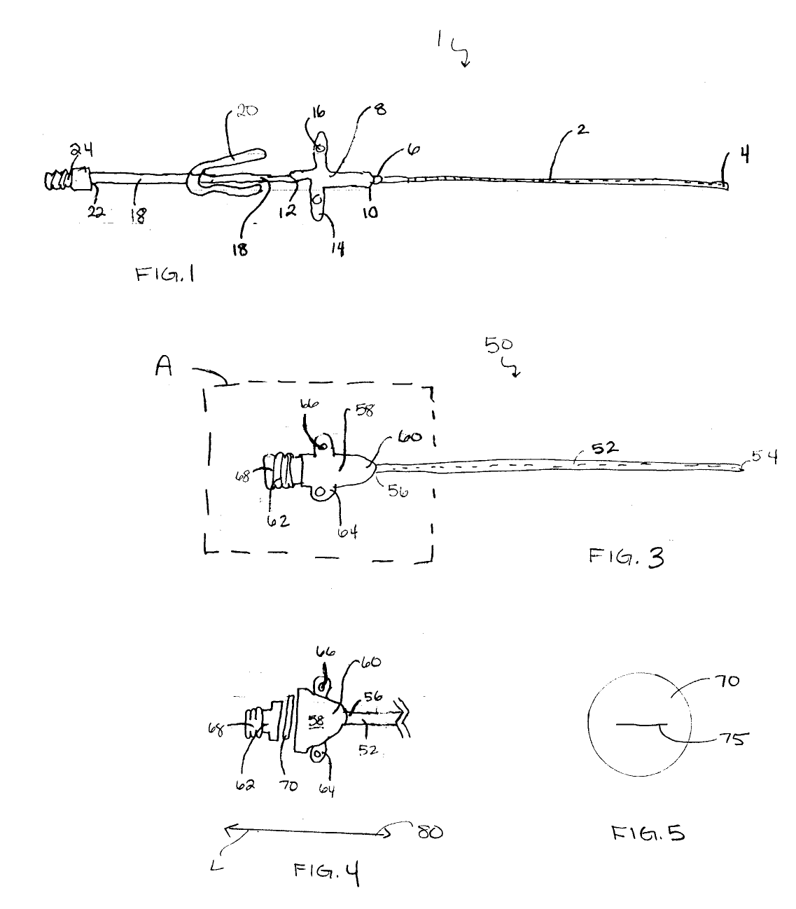

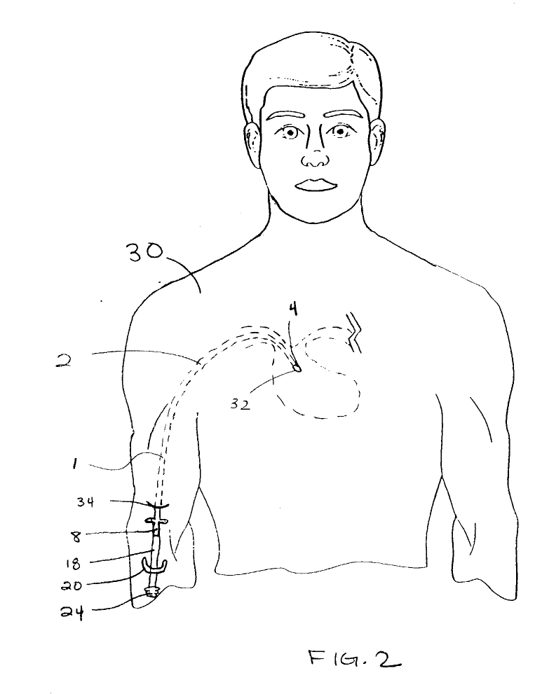

FIG. 3 shows an exemplary embodiment of a PICC 50 manufactured in accordance with the teachings of the present invention. The PICC 50 includes a catheter (distal tubing) 52 and a valved hub 58. The catheter 52 has an open distal end 54, a proximal end 56, and a lumen extending from the proximal end 56 to the open distal end 54.

Attached to the proximal end 56 of the catheter is the valved hub 58. When the PICC 50 is in use, the valved hub 58 remains external to the patient's body. Referring to FIGS. 3-5, the valved hub 58 includes a distal portion 60, a proximal portion 62, and a flexible, thin disk 70 including a slit 75 (slit valve). Each of the distal and proximal portions 60, 62 define a lumen extending therethrough. The proximal portion 62 can include a luer proximal end 68 for attaching the PICC 50 to a fluid introduction source.

Disposed within the valved hub 58 between the distal and proximal portions 60, 62 is the flexible, thin disk 70 including the slit 75. The flexible, thin disk 70 with the slit 75 is used as a valve, which opens and closes depending on a pressure differential across the slit 75. For example, if the pressure differential between the proximal portion 62 and the distal portion 60 is less than a threshold value, the slit 75 within the flexible, thin disk 70 will remain closed, thereby preventing liquid from leaking from the PICC 50. However, if the pressure differential across the flexible, thin disk 70 is greater than the threshold value, the slit 75 will open and fluid can be transported from the luer proximal end 68 to the distal tip 54 or alternatively, liquid can be transported from the distal tip 54 to the luer proximal end 68.

The flexible, thin disk 70 is positioned within the valved hub 58, such that a portion the flexible, thin disk 70 is in contact with the proximal portion 62 and another portion of the flexible, thin disk 70 is in contact with the distal portion 60 of the valved hub 58. In addition, when the flexible, thin disk 70 is positioned within the valved hub 58, the slit 75 is substantially perpendicular to a longitudinal axis L, 80 of the valved hub 58.

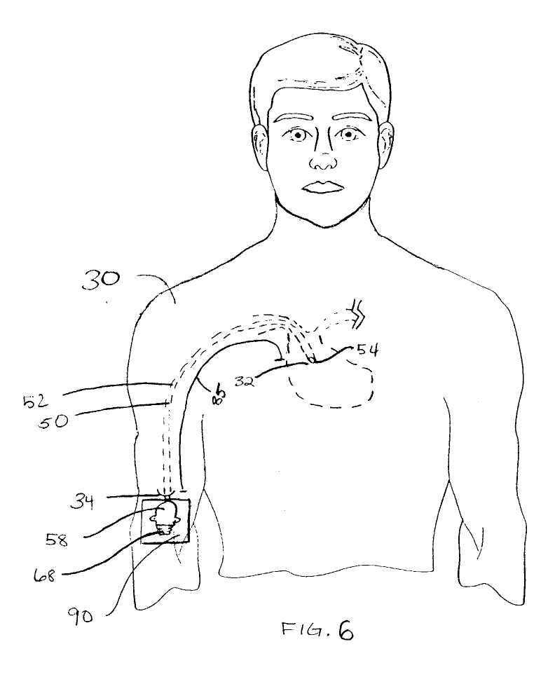

The flexible, thin disk 70 is typically made from silicone, but other biocompatible, flexible, elastomer materials can be used as well. The slit 75, shown in FIG. 5 is a latitudinal cut. However in other embodiments, not shown, the slit 75 can have other configurations, such as an “H” shape, or a sinusoidal shape.

The valved hub 58 can also include a set of suture wings 64 extending off of the distal portion 60. The suture wings 64 are used to secure the hub 58 to the patient's body via suture openings 66. Specifically, the valved hub 58 can be secured by the use of an anchor including two vertical post spaced at a distance corresponding to the distance between the suture openings 66. The anchor is attached to the patient's skin via an adhesive pad. Thus, when the posts of the anchor are inserted into the suture openings 66 the valved hub 58 is secured to the skin via the anchor and adhesive pad.

FIG. 6 shows the PICC 50 after insertion into the patient's body 30. The PICC 50 is inserted into a vein at the entry site 34 located on the patient's right arm. When properly positioned, the distal end 54 of the catheter is located at a predetermined cardiac site, such as, within the right atrium of the patient's heart 32, the catheter 50 is within the patient's vein, and the valved hub 58 is external to the patient's body proximal and immediately adjacent to the entry site 34 (e.g., located less than about 3.81 centimeters from the entry site 34). Thus, the catheter 50 spans a distance 85 commensurate with a distance from the patient's right atrium of the heart 32 to the entry site 34. The entry site 34 shown in FIG. 6 is the patient's right arm, however other possible entry sites include, for example, the groin, neck, and back. The medical professional can measure radiographically the distance 85 prior to inserting the PICC 50 including catheter 52. The medical profession can then either select an appropriately sized PICC 50 having a catheter with a length corresponding to distance 85, or alternatively, the medical profession can trim the catheter 52 to the measured length.

It is important to note that the PICC 50 does not include proximal tubing extending from the valved hub 58, nor does PICC 50 include a clamp disposed on the catheter 52. Thus, the PICC 50 manufactured in accordance with the teachings of the present invention is less likely than the prior art PICC 1 to irritate the patient's skin, because the PICC 50 does not include proximal tubing that extends about 5.08 centimeters (2 inches) to about 20.32 centimeters (8 inches) from the entry site 34. Instead, the PICC 50 terminates at a distance less than about 3.81 centimeters and typically between about 1 centimeters and about 3 centimeters from the entry site 34. In some embodiments, it is preferred that the PICC 50 terminates at a distance less than about 2.54 centimeters from the entry site. Thus, PICC 50 is more tolerable to the patient than the awkward 5.08 centimeter to 20.32 centimeter length of the external portion of prior art PICC 1. In addition, PICC 50 does not include a clamp to impede flow. Instead, the valved hub 58 has an internal valve, the thin, flexible disk 70 including the slit 75, to regulate flow. Since there is no clamp or proximal tubing extending from the valved hub 58, the PICC 50 is less irritating to the patient than a similarly inserted prior art PICC 1.

To supply fluids to the patient, the medical professional attaches the fluid introduction source to the luer proximal end 68 and then releases fluid from the fluid introduction source. The fluid entering into the proximal portion 62 of the valved hub 58 creates a pressure differential across the flexible, thin disk 70 including the slit 75. The slit 75 responds to a pressure differential that is above threshold by opening to allow fluids to pass therethrough and into the lumen of the catheter 52.

In between treatments, the luer proximal end 68 is free of the fluid introduction source and can be covered with a transparent dressing 90. The transparent dressing 90 further secures the valved hub 58 to the patient's body 30, while at the same time covers and protects both the entry site 34 and the valved hub 58 from infection.

Variations, modifications, and other implementations of what is described herein will occur to those of ordinary skill in the art without departing from the spirit and scope of the invention. The invention is not to be limited only to the preceding illustrative description.

What is claimed is:

1. A valved catheter assembly comprising:
a catheter comprising:
a distal end including an opening;
a proximal end; and
at least one lumen extending therethrough; and
a hub attached to the proximal end of the catheter, the hub comprising;
a distal portion defining a lumen extending therethrough;
a proximal portion defining a lumen extending therethrough and including a luer proximal end; and
a flexible, thin disk including a slit, the flexible, thin disk disposed within the hub such that a portion of the flexible, thin disk is in contact with the distal portion of the hub, and another portion of the flexible, thin disk is in contact with the proximal portion of the hub and the slit is substantially perpendicular to a longitudinal axis of the hub;
wherein the valved catheter assembly is sized such that the catheter spans a distance commensurate with a distance from a predetermined cardiac site to an entry site in the patient's skin.

2. The valved catheter assembly of claim 1 wherein the distal portion of the hub includes a suture wing.

3. The valved catheter assembly of claim 1 wherein when the valved catheter assembly is positioned within the patient's body the valved catheter assembly further comprises a transparent dressing.

4. The valved catheter assembly of claim 1 wherein the hub is situated immediately adjacent to the entry site.

5. The valved catheter assembly of claim 1 wherein the hub is secured to the patient's skin.

6. The valved catheter assembly of claim 5 wherein an anchor is used to secure the hub to the patient's skin.

7. The valved catheter assembly of claim 1 wherein the flexible, thin disk comprises silicone.

8. A valved catheter assembly comprising:
a hub comprising a distal end, a luer proximal end, and a valve disposed therebetween; and
distal tubing extending in a distal direction from the distal end of the hub; wherein the luer proximal end of the hub is free of proximal tubing.

9. The valved catheter assembly of claim 8 wherein the distal end of the hub includes a suture wing.

10. The valved catheter assembly of claim 8 wherein when the valved catheter assembly is positioned within a patient's body the valved catheter assembly further comprises a transparent dressing.

11. The valved catheter assembly of claim 8 wherein the hub is secured to a patient's skin.

12. The valved catheter assembly of claim 11 wherein an anchor is used to secure the hub to the patient's skin.

13. The valved catheter assembly of claim 8 wherein the valve comprises a flexible, thin disk including a slit.

14. A valved catheter assembly comprising:
a hub including a slit valve disposed therein; and
a catheter disposed distal to the hub and in fluid communication with the hub, the catheter comprising a distal end, a proximal end, and a tubular element extending between the distal end and proximal end;
wherein the tubular element is free of a clamp disposed thereon.

15. The valved catheter assembly of claim 14 wherein the hub further includes a suture wing.

16. The valved catheter assembly of claim 14 wherein when the valved catheter assembly is positioned within a patient's body the valved catheter assembly further comprises a transparent dressing.

17. The valved catheter assembly of claim 14 wherein the hub is secured to a patient's skin.

18. The valved catheter assembly of claim 17 wherein an anchor is used to secure the hub to the patient's skin.

19. The valved catheter assembly of claim 14 wherein the slit valve comprises a flexible, thin disk.

20. A method of delivering fluids to a patient, the method comprising the steps of:
providing a valved catheter assembly, the valved catheter assembly comprising:
a hub comprising a distal end, a luer proximal end, and a valve disposed therebetween; and;
distal tubing extending in a distal direction from the distal end of the hub;
wherein the luer proximal end of the hub is free of proximal tubing;
inserting the valved catheter assembly into the patient's body through an entry site; and
attaching the luer proximal end to tubing adapted for delivering fluids to the valved catheter assembly.

21. The method of claim 20 further comprising the step of measuring a distance spanning between the patient's right atrium and the entry site and then trimming the distal tubing to a length substantially equal to said distance prior to inserting the valved catheter assembly into the patent's body.
